(19) United States
(12) Patent Application Publication
(10) Pub. No.: US 2025/0241852 A1
(43) Pub. Date: Jul. 31, 2025

(54) PREVENTION OF VISIBLE PARTICLE FORMATION IN AQUEOUS PROTEIN SOLUTIONS

(71) Applicant: Hoffmann-La Roche Inc., Little Falls, NJ (US)

(72) Inventor: Andrea ALLMENDINGER, Grenzach-Wyhlen (DE)

(73) Assignee: Hoffmann-La Roche Inc., Little Falls, NJ (US)

(21) Appl. No.: 19/071,671

(22) Filed: Mar. 5, 2025

Related U.S. Application Data
(63) Continuation of application No. 17/742,625, filed on May 12, 2022.
Application No. 17/742,625 is a continuation of application No. PCT/EP2020/081999, filed on Nov. 13, 2020.

(30) Foreign Application Priority Data
Nov. 15, 2019 (EP) 19209359.9

(51) Int. Cl. A61K 9/107; A61K 9/16; A61K 47/36; A61K 47/42; C07K 16/18

(52) U.S. Cl. CPC A61K 9/107; A61K 9/1617; A61K 47/36; A61K 47/42; C07K 16/18

(57) ABSTRACT

The present invention provides methods to prevent the formation of visible particles in aqueous protein formulations, as well as compositions and pharmaceutical products obtained with said method.

### CROSS REFERENCE TO RELATED APPLICATIONS

This application is a continuation of International Application No. PCT/EP2020/081999, filed Nov. 13, 2020, which claims priority to European Patent Application No. 19209359.9, filed Nov. 15, 2019, which are incorporated herein by reference in its entirety.

The present invention relates to the field of aqueous protein compositions, in particular pharmaceutical antibody formulations, which are stabilized against the formation of visible particles comprising free fatty acids.

### BACKGROUND OF THE INVENTION

Surfactants are crucial excipients in protein formulations as they protect the labile protein from interfacial stress that may lead to protein aggregation. Proteins, such as monoclonal antibodies (mAb), are administered parenterally, which limits the choice of the surfactant, including one of the most commonly used surfactants polysorbate 20 (PS20), but also polysorbate 80, poloxamer 188, and Kolliphor/Solutol® HS 15 (poly-oxyethylene ester of 12-hydroxystearic acid).¹ PS20 can degrade over the shelf-life of a product either by oxidative degradation or by enzymatic, hydrolytic degradation. In particular, the latter yields free fatty acids (FFA) as degradation products, which can precipitate in solution and subsequently form sub-visible and visible particles.² Under conditions typically found in biopharmaceutical formulations, FFA can precipitate even below their solubility limit dependent on temperature but the time point of particle precipitation is poorly understood even for well-characterized degradation profiles. This suggests the involvement of nucleation factors.

There is thus a need to provide efficient solutions to prevent the formation of visible particles in aqueous protein solution, especially for long term storage. The present invention provides mitigation options for FFA particle formation below their solubility limit by selection and treatment of primary packaging material thus reducing the amount of glass leachables acting as nucleation factor. Previous work demonstrated heterogeneity of glass surfaces for single vial lots, which could translate in differences in glass leaching upon storage.³ For this invention, glass leachables were studied as nucleation factors for FFA particle formation.

### BRIEF DESCRIPTION OF THE FIGURES

FIG. 1: Identification of visible particles by FTIR as free fatty acid (FFA) after spiking of myristic acid to glass leachables solution generated from Expansion 51 vials filled with 20 mM glycine solution (pH 10) after three times terminal sterilization. Only a small selection of FFA particles are highlighted.

FIG. 2: Representative FFA particle with Aluminium (few highlighted in dark circles) and Magnesium (dashed circle) on gold filter by SEM-EDX. The chemical composition of the particle is summarized in the table below. The particle was identified after spiking of glass leachables (generated from Exp33 vials with glycine solution) to aged protein solution (22M 5° C.) containing degraded PS20/mixtures of free fatty acids.

FIG. 3: Proposed mechanism of FFA particle formation dependent on nucleation factors exemplarily shown for myristic acid and aluminium.

FIG. 4: Historical real-time glass leachables data from Exp51 vials generated from three different placebo solutions dependent on storage time and vial format justifying leachables concentrations at end of shelf life.

FIG. 5: PS 20 concentration of mAb1 and mAb2 dependent on storage time and temperature.

FIG. 6: Myristic acid (A) and lauric acid (B) concentration of mAb1 and mAb2 dependent on storage time and temperature. The presence of visible particles is indicated by a dashed grey box in the first graph. The samples used for the spiking experiments are indicated by a dashed black box.

### DETAILED DESCRIPTION OF THE INVENTION

Formation of visible particles composing of FFA as a result of surfactant degradation, especially PS20 and/or PS80 degradation, represents a major challenge in the biopharmaceutical industry as there is limited choice for parenteral surfactants. Reducing PS20 degradation and degradation products such as FFA by various means is key as FFA can precipitate above their solubility limit without specific nucleation factors. Below their solubility limit however, nucleation factors can induce precipitation of FFA and limit the shelf life of the product.

Surfactants are essential components in protein formulations protecting against interfacial stress and subsequent protein aggregation. One of the current industry-wide challenges is enzymatic degradation of parenteral surfactants such as polysorbate 20 (PS20), which potentially leads to the formation of free fatty acids (FFA) forming visible particles over the course of the shelf life of a commercial protein containing preparation such as, for example, a commercial aqueous antibody formulation. While concentration of FFAs can reliably be quantified, the time point of particle formation in solution stored in glass vials remains unpredictable. The present inventors therefore studied the influence of inorganic ions, such as glass leachables, for example, as nucleation factors for FFA particle formation.

Table A, below, summarizes the concentrations of the most relevant glass leachables for different primary packaging material depending on the stored solution, preparation of glass material (e.g., terminal sterilization), solution storage time, and temperature clearly highlighting the reduction of leachables for surface-modified glass vials in comparison to uncoated glass vials (Exp33/Duran® and Exp51/Fiolax® vials). Among the surface modified vials are Siliconized vials, TopLyo® vials (Si—O—C—H layer, https://www.schott.com/d/pharmaceutical_packaging/7f629b7e-e978-4417-a15e-8621f969d225/1.4/schott-datasheet-schott-toplyo-english-14062017.pdf), and

Type I plus® vials (covalently bound SiO₂ layer, https://www.schott.com/d/pharmaceutical_packaging/ff592e9e-4a7f-495f-9952-965c4d7bled8/1.4/schott-datasheet-schott-type-i-plus-english-14062017.pdf).

TABLE A
Concentration of glass leachables Aluminium, Boron,
Silicon, and Sodium in different glass types with different solutions dependent on storage
temperature, time, and glass preparation. Concentrations are compared to the initial
concentration of glass leachables of the solutions and their limit of quantification (LOQ).

| Time | | | Elemental concentration (ug/mL) | | | |
|---|---|---|---|---|---|---|
| point | Vial type | Matrix | Al | B | Si | Na |
| Initial | — | WFI | 0.001 | 0.012 | <LOQ | 0.095 |
| | | Glycine pH 10 | 0.057 | 0.18 | 1 | n.m. |
| | | Placebo | <LOQ | <LOQ | <LOQ | <LOQ |
| | Exp33 | WFI | <LOQ | 0.16 | 0.2 | 0.23 |
| | | Glycine pH 10 | 0.053 | 0.34 | 2.7 | n.m. |
| | | Placebo | <LOQ | 0.26 | <LOQ | <LOQ |
| | | 1xTS Glycine | 0.24 | 1.5 | 12 | n.m. |
| | | pH 10 | | | | |
| | | 1xTS Placebo | <LOQ | 0.82 | <LOQ | 1 |
| | Exp51 | WFI | <LOQ | 0.028 | 0.05 | 0.2 |
| | | Glycine pH 10 | 0.065 | 0.19 | 1.7 | n.m. |
| | | Placebo | <LOQ | 0.09 | <LOQ | <LOQ |
| | | 1xTS Glycine | 0.37 | 0.43 | 4.3 | n.m. |
| | | pH 10 | | | | |
| | | 1xTS Placebo | 0.026 | 0.22 | <LOQ | 1.1 |
| | TopLyo ® | WFI | <LOQ | <LOQ | 0.13 | 0.018 |
| | | Glycine pH 10 | 0.046 | 0.17 | 2.3 | n.m. |
| | | Placebo | <LOQ | <LOQ | <LOQ | <LOQ |
| | | 1xTS Glycine | 0.045 | 0.17 | 2 | n.m. |
| | | pH 10 | | | | |
| | | 1xTS Placebo | <LOQ | 0.05 | <LOQ | <LOQ |
| | Siliconized | WFI | 0.002 | 0.066 | 0.07 | 0.2 |
| | | Glycine pH 10 | 0.063 | 0.23 | 1.8 | n.m. |
| | | Placebo | <LOQ | 0.12 | <LOQ | <LOQ |
| | | 1xTS Glycine | 0.25 | 0.42 | 3.6 | n.m. |
| | | pH 10 | | | | |
| | | 1xTS Placebo | <LOQ | 0.2 | <LOQ | 0.93 |
| | Type I plus ® | WFI | <LOQ | <LOQ | 0.06 | 0.016 |
| | | Glycine pH 10 | 0.033 | 0.17 | 1.8 | n.m. |
| | | Placebo | <LOQ | 0.05 | <LOQ | <LOQ |
| | | 1xTS Glycine | 0.028 | 0.22 | 9.2 | n.m. |
| | | pH 10 | | | | |
| 6M 40° C. | Exp33 | 1xTS Placebo | <LOQ | 0.056 | <LOQ | <LOQ |
| | | WFI | 0.002 | 1 | 2.2 | 0.78 |
| | | Glycine pH 10 | 0.019 | 12 | 67 | n.m. |
| | | Placebo | 0.055 | 1.1 | 1.7 | 1.1 |
| | | 1xTS Glycine | 0.017 | 12 | 69 | n.m. |
| | | pH 10 | | | | |
| | | 1xTS Placebo | 0.09 | 1.3 | 2.8 | 1.2 |
| | Exp51 | WFI | 0.026 | 0.1 | 0.73 | 0.55 |
| | | Glycine pH 10 | 0.97 | 0.97 | 8.8 | n.m. |
| | | Placebo | 0.12 | 0.2 | 1 | 0.66 |
| | | 1xTS Glycine | 0.99 | 1 | 8.9 | n.m. |
| | | pH 10 | | | | |
| | | 1xTS Placebo | 0.21 | 0.28 | 1.6 | 1.1 |
| | Toplyo ® | WFI | <LOQ | <LOQ | 1.2 | 0.012 |
| | | Glycine pH 10 | 0.054 | 0.17 | 3.2 | n.m. |
| | | Placebo | <LOQ | <LOQ | 0.59 | <LOQ |
| | | 1xTS Glycine | 0.05 | 0.17 | 3.5 | n.m. |
| | | pH 10 | | | | |
| | | 1xTS Placebo | 0.068 | <LOQ | 0.76 | <LOQ |
| | Siliconized | WFI | 0.018 | 0.2 | 1.1 | 0.67 |
| | | Glycine pH 10 | 0.79 | 0.99 | 9.1 | n.m. |
| | | Placebo | 0.069 | 0.18 | 0.79 | 0.58 |
| | | 1xTS Glycine | 0.81 | 0.98 | 9 | n.m. |
| | | pH 10 | | | | |
| | | 1xTS Placebo | 0.16 | 0.28 | 1.4 | 0.96 |
| | Type I plus ® | WFI | <LOQ | <LOQ | 0.5 | 0.014 |
| | | Glycine pH 10 | 0.15 | 0.3 | 18 | n.m. |
| | | Placebo | <LOQ | <LOQ | 5.6 | <LOQ |
| | | 1xTS Glycine | 0.19 | 0.41 | 19 | n.m. |
| | | pH 10 | | | | |
| | | 1xTS Placebo | <LOQ | <LOQ | 2.3 | <LOQ |
| LOQ | — | WFI | 0.001 | 0.005 | 0.05 | 0.005 |
| | | Glycine pH 10 | 0.01 | 0.1 | 0.5 | n.m. |
| | | Placebo | 0.02 | 0.05 | 0.5 | 0.5 |

TS = terminal sterlized,
n.m. = not measured

In accordance with the present invention, it has now been found that FFA particles, identified by FTIR, were a result of precipitation with inorganic ions/components, e.g. glass leachables such as aluminium, as elucidated by further characterization of the chemical composition by SEM-EDX. This suggests the involvement of inorganic elements in the formation of these particles. Silicon dioxide, boron trioxide, and aluminium trioxide are typical glass network formers of type I borosilicate glass used for parenteral products. Different glass network modifiers like alkali oxides (e.g., sodium, potassium), and oxides of alkaline earth metals (e.g., calcium and magnesium) are added during the glass manufacturing process to decrease the melting temperature of the glass. Without being bound to theory, it may be concluded that inorganic elements leaching from the glass vials depending on glass type, formulation, and storage condition may act as nucleation seeds for FFA particle formation. In the present study, lauric acid and myristic acid were used as main degradation products from enzymatic PS20 degradation⁴ and the study targeted different glass leachables as well as mixtures of the same to verify the hypothesis that free fatty acids below their solubility limit precipitate in their presence.

In accordance with the present invention, it has surprisingly been found that inorganic salts especially NaAlO₂ and CaCl₂ initiate the formation of visible particles in presence of myristic or lauric acid below their solubility limit. These salts mimic leachables from type I borosilicate glass typically used for parenteral products. In particular surprisingly, relevant glass leachables in mixtures obtained by autoclavation cycles in different glass types with different formulations and at representative leachable's concentrations for a proteinaceous drug product over its shelf life of 2-3years at 5° C. confirmed particle formation with lauric/myristic acid the major degradation products of polysorbates, such as PS20, in commercial parenteral antibody formulations. Particles in different formulations in Exp33 vials and Exp51 vials were identified as FFA salts with different glass leachables, such as aluminium or silicon. In addition, the present invention in particular demonstrated FFA particle formation depending on relevant aluminum concentrations. In one embodiment in accordance with the present invention, said aluminium concentrations are in the ppb range. The present findings were verified in two case studies with monoclonal antibody (mAb) formulations aged at recommended storage temperature (22 M, 5° C.) showing enzymatic PS20 degradation profiles resulting in mixtures of different FFA. In these, spiking of mixtures of glass leachables led to immediate visible particle formation, identified as a complex of glass leachables such as aluminium, silicon, magnesium, potassium, sodium, calcium and free fatty acid. Based on the present results, particle formation will be verified in protein formulations on long-term storage under real-time conditions.

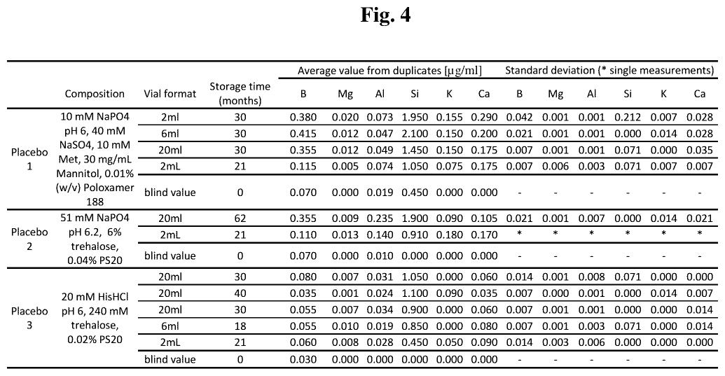

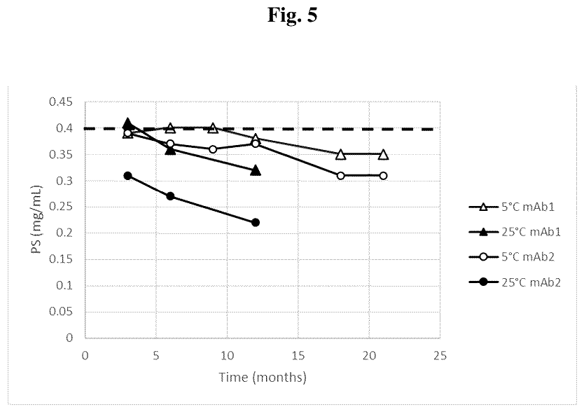

Therefore, in one embodiment the present invention provides a stable aqueous composition comprising a protein together with pharmaceutically acceptable excipients such as, for example, buffers, stabilizers including antioxidants, and surfactants, wherein said composition further contains a mixture of one or several types of inorganic ions diffused out of the packaging material, such as a glass vial, and substances resulting from the degradation of said surfactants without forming visible particles. In one aspect said inorganic ions are selected from Aluminium, Boron, Silicon, Calcium, Magnesium, Potassium, and Sodium. In another aspect the concentration of said inorganic ions is any concentration up to the concentration disclosed in Table A (6 M 40° C.) for each ion and each vial type for non-surface-modified vials (referred to as Exp33 and exp51 vials), respectively. In yet another aspect, specifically for Exp51 vials of different vial formats, the concentration of said inorganic ions is any concentration up to the respective concentrations for each ion as disclosed in FIG. 4.

In another embodiment, there is provided the composition as defined above, wherein the pH of said composition is in the range of 5 to 7. In one aspect the pH is about 6.

In another embodiment, the present invention provides a composition as defined herein before, wherein the protein is an antibody. In one aspect, the antibody is a monoclonal antibody. In another aspect the antibody is a human or humanized monoclonal, mono- or bispecific antibody.

In one aspect, the present invention provides a composition as defined herein before, further comprising one or several types of substances resulting from the degradation of surfactants present in said composition (degradation products). In one aspect said surfactant is selected from polysorbates (PS). In another aspect, said surfactant is selected from PS20 or PS80. In another aspect, said degradation products are a mixture of different fatty acids of different chain length and saturation and remaining PS20 residues consisting of polymeric esters of different polar head groups, different fatty acid tails, and different degree of esterification. In one aspect said degradation products are free fatty acids as defined herein. In one aspect, said substances resulting from polysorbate degradation are free fatty acids in a concentration up to, but not above, their respective solubility level. In another aspect, said free fatty acid is selected as defined in USP in PS20. In another aspect, said free fatty acid is selected from lauric acid, myristic acid, palmitic/oleic acid, capric acid, and stearic acid. In another aspect, said free fatty acid is selected from lauric acid and/or myristic acid and the solubility level for lauric acid is 15 μg/ml, and the solubility level for myristic acid is 7 μg/ml in water at room temperature.

In one embodiment, the present invention provides a composition as defined herein before, wherein the concentration is up to 0.03 μg/ml aluminium, and/or up to 0.05 μg/ml boron, and/or up to 0.5 μg/ml silicon.

In another embodiment, the present invention provides a composition as defined herein before, wherein the stabilizer is selected from the group consisting of sugars, sugar alcohols, sugar derivatives, or amino acids. In one aspect the stabilizer is (1) sucrose, trehalose, cyclodextrines, sorbitol, mannitol, glycine, or/and (2) methionine, and/or (3) arginine, or lysine. In still another aspect, the concentration of said stabilizer is (1) up to 500 mM or (2) 5-25 mM, or/and (3) up to 350 mM, respectively.

In another embodiment, the present invention provides a composition as defined herein before, wherein the buffer is selected from the group consisting of acetate, succinate, citrate, arginine, histidine, phosphate, Tris, glycine, aspartate, and glutamate buffer systems. In one aspect the buffer composes of free histidine base and histidine-HCl or acetate or succinate and/or aspartate. Furthermore, within this embodiment, the histidine concentration of said buffer is from 5 to 50 mM.

In another embodiment, the present invention provides a composition as defined herein before, wherein the surfactant is selected from the group consisting of non-ionic surfactants. In one aspect, the surfactant is a polysorbate (PS). In another aspect, the surfactant is PS20 or PS80 or Polyoxyl 15 Hydroxystearate. In yet another aspect, the concentration of said surfactant is from 0.01%-1% (w/v).

In another embodiment, the present invention provides a composition as defined herein before, wherein the pharmaceutically acceptable excipients are: 1000 U/mL hyaluronidase in 20 mM HisHCl buffer pH 5.5, 105 mM Trehalose, 100 mM Sucrose, 10 mM Methionine, and 0.04% (w/v) Polysorbate 20.

In another embodiment, the present invention provides a composition as defined herein before, characterized in that it remains free of visible particles. In one aspect said visible particles consist of degradation products and inorganic ions, as defined herein. In one aspect said visible particles consist of free fatty acids and inorganic ions, as defined herein.

In another embodiment, the present invention provides a composition as defined herein before, wherein said composition remains free of said visible particles until the end of its authorized shelf life. In another aspect said composition remains free of said visible particles for up to 5 years, or for up to 3 years, or for up to 24 months, or for up to 18 months, or for up to 12 months.

In another embodiment, the present invention provides a method for obtaining a composition as defined herein, wherein said method comprises selecting a primary packaging material which prevents leaching of one or several inorganic ions as defined herein into said composition. In one aspect, said method prevents leaching of said one or several inorganic ions above the respective concentration given in Table A (6M 40° C., non-surface-modified vials) and/or FIG. 4. In another embodiment the present method prevents leaching of up to 0.03 μg/ml aluminium, and/or up to 0.05 μg/ml boron, and/or up to 0.5 μg/ml silicon.

In one embodiment, the present invention provides the method for obtaining a composition as defined herein, wherein said primary packaging material is selected from
Glass vials with inner surface coating
Glass vials with covalently modified surface
Glass vials from pure SiO₂ (>99%)
Glass vials that are washed and sterilized as described below
Polymer vials
Polymer vials with inner surface coating or surface modification

In another embodiment, the present invention provides the method for obtaining a composition as defined herein, wherein said primary packaging material is selected from
Siliconized vials,
TopLyo® vials,
Type I plus® vials,
Pur Q® vials,
Crystal Zenith® vials,
SiO₂ material sciencesm vials,
Duran® vials washed and sterilized as described below, and/or
Fiolax® vials washed and sterilized as described below.

In another embodiment, the present invention provides the method for obtaining a composition as defined herein, further comprising the steps of a) washing/drying and/or b) depyrogenation of the primary packaging material prior to its use, for example, prior to filling in the aqueous protein composition. In one aspect the washing is carried out at water temperatures above 50° C. followed by a drying step allowing for residual water of <50 μl. In one aspect the depyrogenation is carried out at temperatures below or equal to 400° C. In another aspect the depyrogenation is carried out at temperatures between 180-340° C. and residence time in the sterilization tunnel is limited to 8 h.

In another embodiment, the present invention provides the method for obtaining a composition as defined herein, wherein said method provides stability of said composition against the formation of visible particles. In one aspect said visible particles comprise one or several degradation products and one or several types of inorganic ions as defined herein. In another aspect said visible particles consist of one or several free fatty acids and one or several inorganic ions, as defined herein. In yet another aspect, the method in accordance with the present invention provides a composition, for example a commercial pharmaceutical antibody composition, which remains free of visible particles until the expiry of its authorized shelf life. In another aspect the present method provides a composition which remains free of said visible particles for up to 5 years, or for up to 3 years, or for up to 24 months, or for up to 18 months, or for up to 12 months.

In another embodiment, the present invention provides a pharmaceutical dosage form comprising a composition as defined herein, for example an aqueous antibody composition, in a container, wherein the concentration of one or several inorganic ions in that composition remains substantially constant during the authorized shelf life of said pharmaceutical dosage form. In one aspect said concentration of one or several inorganic ions remains substantially constant for up to 5 years, or 3 years, or 24 months, or 18 months, or 12 months of storage, when compared to the concentration(s) of the same ion(s) measured in a pharmaceutical dosage form comprising the same composition in the same container at the beginning of storage, for example after 2 weeks, or immediately after filling said composition into said container or packaging material. In one aspect the container is a glass vial or a primary packaging material as defined herein. In one aspect the inorganic ions are selected from Aluminium, Boron, Silicon, Calcium, Magnesium, Potassium, and Sodium.

In another embodiment, the present invention provides the pharmaceutical dosage form as defined herein before, for example an aqueous antibody formulation in a container, wherein the increase in concentration of one or several inorganic ions in said dosage form remains below the respective concentration given for each ion and each vial type in Table A (non-surface modified vials, 6 M 40° C.) and/or FIG. 4. In another aspect, and independent of the vial type, the concentration of aluminium remains below 0.03 μg/ml, and/or the concentration of boron remains below 0.05 μg/ml, and/or the concentration of silicon remains below 0.5 μg/ml after up to 5 years, or 3 years, or 24 months, or 18 months, or 12 months of storage when compared to the concentration(s) of the same ion(s) measured in a pharmaceutical dosage form comprising the same composition in the same container at the beginning of storage, for example after 2 weeks, or immediately after filling said composition into said container or packaging material. In one aspect the inorganic ions are selected from Aluminium, Boron, Silicon, Calcium, Magnesium, Potassium, and Sodium. In one aspect the container is a glass vial, or a primary packaging material as defined herein.

In another embodiment, the present invention provides the use of a “primary packaging material”, as defined herein before, for the storage of aqueous antibody preparations. In another embodiment, the present invention provides the use of said “primary packaging material”, as defined herein, to reduce or avoid formation of visible particles, for example particles comprising FFA's, during storage of aqueous antibody preparations. In one embodiment, said primary packaging material is a polymer vial, as defined herein. In another embodiment, said primary packaging material is a surface modified glass vial as defined herein. In still another embodiment, said antibody is a monoclonal antibody. In another embodiment, said storage is characterized in that said antibody preparation remains free of visible particles for at least the authorized shelf life of the corresponding antibody product. In another embodiment said storage is characterized in that said antibody preparation remains free of visible particles for up to 5 years, or 3 years, or 24 months, or 18 months, or 12 months of storage.

The term “excipient” refers to an ingredient in a pharmaceutical composition or formulation, other than an active ingredient, which is nontoxic to a subject. An excipient includes, but is not limited to, a buffer, stabilizer including antioxidant, surfactant, or preservative.

The term “inorganic ions” is well known to a person of skill in the art of inorganic chemistry. Inorganic ions as used herein means aluminium, boron, silicon, sodium, magnesium, potassium, and calcium. Preferred inorganic ions are Aluminium, calcium, and magnesium. In accordance with the present invention, said inorganic ions can be present in a concentration of up to 0.03 μg/ml aluminium, and/or up to 0.05 μg/ml boron, and/or up to 0.5 μg/ml silicon.

The term “buffer” is well known to a person of skill in the art of organic chemistry or pharmaceutical sciences such as, for example, pharmaceutical preparation development. Buffer as used herein means acetate, succinate, citrate, arginine, histidine, phosphate, Tris, glycine, aspartate, and glutamate buffer systems. Furthermore, within this embodiment, the histidine concentration of said buffer is from 5 to 50 mM. Preferred buffers are free histidine base and histidine-HCl or acetate or succinate and/or aspartate. Furthermore, within this embodiment, the histidine concentration of said buffer is from 5 to 50 mM.

The term “surfactant” is well known to a person of skill in the art of organic chemistry. Surfactants as used herein means non-ionic surfactants. Preferred surfactants are polysorbates, especially PS20 or PS80. In accordance with the present invention, said surfactant can be present in a concentration from 0.01%-1% (w/v).

The term “stabilizer” is well known to a person of skill in the art of organic chemistry or pharmaceutical sciences such as, for example, pharmaceutical preparation development. A stabilizer in accordance with the present invention is selected from the group consisting of sugars, sugar alcohols, sugar derivatives, or amino acids. In one aspect the stabilizer is (1) sucrose, trehalose, cyclodextrines, sorbitol, mannitol, glycine, or/and (2) methionine, and/or (3) arginine, or lysine. In still another aspect, the concentration of said stabilizer is (1) up to500 mM or (2) 5-25 mM, or/and (3) up to 350 mM, respectively

The term “substances resulting from the degradation of polysorbates” or “degradation products” as used herein means any substance resulting from the degradation of polysorbates know to the skilled person. In one aspect, said substances are free Fatty Acids. The term “Fatty Acid” (or “FA”) is well known to a person of ordinary skill in organic chemistry. In one aspect, fatty acids means any carboxylic acid with an aliphatic chain, which is saturated or unsaturated, linear or branched and contains from 4 to 28; or from 8 to 24; or from 10 to 22; or from 12 to 20 carbon atoms. In one aspect, said free fatty acid is selected as defined in USP in PS20. In one aspect, said free fatty acid is selected from lauric acid, myristic acid, palmitic/oleic acid, capric acid, and stearic acid. In another aspect, said free fatty acid is selected from lauric acid and/or myristic acid. In accordance with the present invention, said substances resulting from the degradation of polysorbates can be present in a concentration up to their respective solubility level in water at room temperature. In another aspect such substances are present at any concentration up to but not including their solubility level in water at room temperature. The term “room temperature” as used herein has its ordinary meaning. In one aspect room temperature means from 20 to 28, preferably from 22 to 26 degrees Celsius.

The term “packaging material” or “primary packaging material” as used herein means material in contact with the product. In one embodiment the term primary packaging material means
Glass vials with inner surface coating,
Glass vials with covalently modified surface,
Glass vials from pure SiO₂ (>99%),
Glass vials that are washed and sterilized as described below,
Polymer vials,
Polymer vials with inner surface coating or surface modification,

In one embodiment the term primary packaging material means
Siliconized vials,
TopLyo® vials,
Type I plus® vials,
Pur Q® vials,
Crystal Zenith® vials,
SiO₂ material science_{SM} vials,
Duran® vials washed and sterilized as described below, and/or
Fiolax® vials washed and sterilized as described below

In certain embodiments, the packaging material is washed and/or depyrogenated prior to receiving said stable aqueous protein composition. Washing of said packaging material can be carried out by any means known to the skilled person. Preferably said washing is carried out using at water temperatures above 50° C. followed by a drying step allowing for residual water of <50 uL. Depyrogenation of said packaging material can be carried out by any means known to the skilled person. Preferably said depyrogenation is carried out using at temperatures below or equal to 400° C. More preferably said depyrogenation is carried out at temperatures between 180-340° C. and residence time in the sterilization tunnel is limited to 8 h.

The term “protein” as used herein means any therapeutically relevant polypeptide. In one embodiment, the term protein means an antibody. In another embodiment, the term protein means an immunocunjugate.

The term “antibody” herein is used in the broadest sense and encompasses various antibody classes or structures, including but not limited to monoclonal antibodies, polyclonal antibodies, multispecific antibodies (e.g., bispecific antibodies), and antibody fragments so long as they exhibit the desired antigen-binding activity. In one combodiment, any of these antibodies is human or humanized. In one aspect, the antibody is selected from alemtuzumab (LEMTRADA®), atezolizumab (TECENTRIQ®), bevacizumab (AVASTIN®), cetuximab (ERBITUX®), panitumumab (VECTIBIX®), pertuzumab (OMNITARG®, 2C4), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F (ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called a, d, e, g, and m, respectively. The light chain of an antibody may be assigned to one of two types, called kappa (κ) and lambda (λ), based on the amino acid sequence of its constant domain.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007). The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to an excipient as defined herein.

A. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al.,

Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### B. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat.

Biotech. 23:1117-1125 (2005). See also, e.g., U.S. Pat. Nos. 6,075,181 and 6,150,584 describing XENOMOUSETM technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### C. Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### D. Immunoconjugates

The invention also provides immunoconjugates comprising an antibody herein conjugated (chemically bound) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above.

The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available (e.g., from Pierce Biotechnology, Inc., Rockford, IL., U.S.A).

### E. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (sce, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to two different antigens, or two different epitopes of the same antigen (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CHI/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

### F. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. No. 5,648,237, U.S. Pat. No. 5,789,199, and U.S. Pat. No. 5,840,523. (See also Charlton, K.A., In: Methods in Molecular Biology, Vol. 248, Lo, B.K.C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T.U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIESTM technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F.L. et al., J. Gen Virol. 36 (1977) 59-74); baby hamster kidney cells (BHK); mouse sertoli cells (TM4 cells as described, e.g., in Mather, J.P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J.P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A.M., Methods in Molecular Biology, Vol. 248, Lo, B.K.C. (ed.), Humana Press, Totowa, NJ (2004), pp. 255-268.

The invention will now be further illustrated by the following, non-limiting working examples.

### EXAMPLES

### Material and Methods

### FFA Solutions (Stock Solutions)

Aqueous stock solutions were prepared in 0.02% PS20 (Croda, Edison, NJ, USA) at defined concentrations of (1) 5 mg/mL and (2) 12.5 mg/mL (lauric acid, Sigma-Aldrich/Merck, Darmstadt, DE) or (1) 1.5 mg/mL and (2) 5 mg/mL (myristic acid, Sigma-Aldrich/Merck, Darmstadt, DE) as previously described by Doshi et al.⁶ The procedure was adapted that the FFA/PS20 stock solution was sterile filtered using 0.22 μm PVDF Steriflip filters (Merck Millipore, Darmstadt, DE) before 1:10 dilution and subsequently homogenized without a magnetic stirrer using a Heidolph Rotamax 120 orbital shaker (Schwabach, DE) at 100 rpm at 25° C. for 1 hour. After 1:500 dilution, the solution was homogenized for 1 hour at 25° C. before homogenization at 2-8° C. overnight. 12-40 μL of stock solution were used for spiking experiments yielding final FFA concentrations of (1) 10 μg/mL and (2) 25 μg/mL (lauric acid) or (1) 3 μg/mL and (2) 10 μg/mL (myristic acid). Dilutions with stock solutions 1 yield FFA concentration below their solubility limit, whereas stock solutions 2 act as positive control confirmed by the formation of visible particles. FFA concentrations were verified for selected by LC-MS samples acc. Honemann et al.⁷

### Inorganic Salt Solutions

Aqueous stock solutions of different salts were prepared at 1 mg/mL and used for spiking experiments to final concentrations between 250 mg/mL and 1 g/mL. NaCl, NaAlO₂, NaBO₂, B₂O₃, and CaCl₂ (Sigma-Aldrich/Merck, Darmstadt, DE) were selected as their dissolution products (ions) represent typical glass leachables from Type I borosilicate glass. The pH of the NaAlO₂ and NaBO₂ stock solutions was adjusted to pH 6 using HCl and subsequently filtered using 0.22 μm PVDF Sterivex filters (Merck Millipore, Darmstadt, DE) and true elemental concentrations were determined by inductively-coupled plasma mass spectrometry (ICP-MS). Elemental concentrations were 0.048 μg/mL Aluminium and 295 μg/mL sodium and 78 μg/mL boron and 168 μg/mL, respectively. Spiking experiments with FFA were performed in duplicates.

### Glass Leachable's Solutions

Representative mixtures of glass leachables were obtained from three different types of glass vials, e.g. Exp 33 and Exp51 (Schott AG, Müllheim, DE, and Schott North America Inc., NY, USA) in the 6 mL format, by three autoclavation cycles (121° C., 20 min) representing accelerate aging conditions. Vials were filled with 6 mL of either water for injection (WFI), 20 mM glycine solution pH 10 or a typical placebo solution used for protein formulations consisting of 20 mM Histidine/Histdine-HCl buffer pH 6.0, 10 mM Methionine, 240 mM sucrose (Ferro Pfanstiehl, Waukegan, IL, USA), and 0.02% PS20. The pH of the glycine solutions was adapted after autoclavation with HCl to pH 6.0 and filtered through a 0.22 μm PVDF Sterivex filters (Merck Millipore, Darmstadt, DE) Leachable concentrations were verified ICP-MS (Table 1) as described by Ditter et al.⁸ Spiking experiments with FFA were performed in triplicates.

TABLE 1
Concentration of selected glass leachables in spiking solutions.

| Vial | | Elemental concentration (μg/mL) | | | | | |
|---|---|---|---|---|---|---|---|
| type | Matrix | Al | B | Si | Na | Ca | K |
| | 3xTS WF | <LOQ | 1 | 0.31 | 1.10 | <LOQ | 0.05 |
| Exp33 | 3xTS | <LOQ | 0.25 | <LOQ | 0.78 | <LOQ | <LOQ |
| | Placebo | | | | | | |
| | 3xTS | 0.56 | 4.7 | 33 | 361 | <LOQ | 1.1 |
| | Glycine | | | | | | |
| | pH 6 | | | | | | |
| | 3xTS WF | 0.08 | 0.12 | 1.4 | 0.80 | 0.06 | <LOQ |
| Exp51 | 3xTS | 0.03 | <LOQ | 0.5 | 2.70 | 0.09 | <LOQ |
| | Placebo | | | | | | |
| | 3xTS | 0.02 | 1.9 | 19 | 397 | 0.28 | 0.71 |
| | Glycine | | | | | | |
| | pH 6 | | | | | | |
| | 3xTS WFI | 0.05 | 0.05 | 0.05 | 0.1 | 0.05 | 0.05 |
| LOQ | 3xTS | 0.01 | 0.2 | 0.5 | 0.3 | 0.05 | 0.2 |
| | Placebo | | | | | | |
| | 3xTS | 0.01 | 0.1 | 0.5 | 0.5 | 0.1 | 0.1 |
| | Glycine | | | | | | |
| | pH 6 | | | | | | |

mAB Formulations mAb1 (IgG₁, Mw=145.5 kDa, pertuzumab) and mAb2 (IgG₁, Mw=148 kDa, trastuzumab) were obtained from F. Hoffmann-La Roche and formulated with 1000 U/mL hyaluronidase in 20 mM HisHCl buffer pH 5.5, 105 mM Trehalose, 100 mM Sucrose, 10 mM Methionine, and 0.04% Polysorbate 20. The corresponding placebo was the same formulation without proteins. Formulations were stored at 5° C. and 25° C. for 24 months. Spiking experiments including respective controls with placebo were performed in triplicates using either inorganic salt solutions (CaCl₂ and NaAlO₂ in 10+1 dilution) or glass leachable's from stock solutions in 10+1 or 100+1 dilution.

### Analytical Characterization

Samples were analyzed by visual inspection on a Seidenader V 90-T instrument (Seidenader Maschinenbau GmbH, Markt Schwaben, DE) as previously described by Ditter et al.⁹ and using a black/white panel according Ph. Eur. 2.9.20.¹⁰ and classified as Many particles (>7), Few particles (4-7), or Practically free of particles (0-4) in E/P box, and Many particles (>10), Few particles (6-10), Essentially free of particles (1-5), or Free of particles (0) by Seidenader.

Sub-visible particles (SVP) were determined by light obscuration according Ph. Eur. 2.9.19.¹¹ using a HIAC/ROYCO 9703 Liquid Syringe Sampler 3000A with a HRLD-150 sensor (Skan AG, Allschwill, CH) as previously described by Ditter et al.⁹

Turbidity was determined as outlined in Ph. Eur. 2.2.1.¹² using a Hach 2100AN turbidimeter (Hach Company, Loveland, Co) in the ratio mode.

Particles >20 μm were further identified by Fourier transformed infrared spectroscopy (FTTIR) using a Nicolet™ iNTM10 Infrared Microscope (Thermo Fisher Scientific) by comparison to reference spectra. Samples were filtrated under laminar air flow through gold-coated polycarbonate filters (pore size 0.8 μm, diameter 13 mm, Sterlitech). Filter conditioning included few droplets of ethanol followed by 1 mL of particle-free water. After filtration of the samples, ˜1 mL of cooled particle-free water was used as a final washing step before analysis.

Chemical compositions of selected particles was verified by Scanning electron microscopy associated with Energy Dispersive X-ray spectroscopy (SEM-EDX) using a Phenom XL instrument from LOT Quantum Design GmbH.

pH of all solutions was verified. Samples were visually inspected immediately after spiking and regularly for up to 7 days. Further characterization by HIAC, turbidity, FTIR, and SEM-EDX was performed on day 1 only. All samples were analyzed when equilibrated to room temperature (4h).

Polysorbate content for the mAb samples was determined by mixed mode HPLC using evaporative light scattering detection.

### Example 1 Artificial Glass Leachables (Salts) Lead to FFA Particle Formation

Different inorganic salt solutions, i.e. CaCl₂, NaAlO₂, NaBO₂, B₂0₃, and NaCl, were prepared simulating artificial glass leachables. Myristic and lauric acid, as main degradation product from hydrolytic PS20 degradation, were added at concentrations below their solubility limit and samples were analyzed for visible particles, SVP, and turbidity. Samples were compared against relevant controls and the pH verified at pH 6.

Immediate formation of visible particles after spiking was observed for both myristic and lauric acid with NaAlO₂ depending on the salt concentration. Immediate particle formation was in particular seen directly after spiking with myristic acid with CaCl₂ for all salt concentrations tested. Increasing particle formation correlated in general with increasing incubation time for both fatty acid solutions as well as for with increasing NaAlO₂ and CaCl₂ concentrations, respectively. Particles were even visible in E/P box in particular for myristic acid in presence of Calcium as summarized in Table 1 depending on the time point of inspection. Particles were subsequently identified by FTIR as FFA particles (data not shown). For NaBO₂ and B₂0₃ solutions, visible particles were obtained in Seidenader for both myristic and lauric acid dependent on the salt concentration over time, but to a much lesser extent compared to CaCl₂ and NaAlO₂. No particle formation was observed when adding sodium chloride up to a salt concentration of 1 mg/mL.

The spiking experiments demonstrate feasibility of FFA particle formation in the visible range in presence of salts simulating relevant glass leachables from borosilicate glass typically used as primary packaging for parenteral products. Particle formation was found to be highly dependent on type and concentration of the ion/salt, like Ca²⁺ or Al³⁺, as well as on incubation time. Besides adequate controls, the time point of inspection and equilibration of the samples/temperature of the solution are crucial for these experiments: FFA solubility is highly dependent on temperature and more particles are detected at lower temperatures, e.g., equilibration time to room temperature of 1 versus 4 h. The solubility limit of FFA is also heavily dependent on pH. Thus, experiments were performed at pH 6. However, NaAlO₂ and NaBO₂ initially form hydroxides in solution (˜pH 10). Thus, the pH of the spiking solutions need to be adjusted and the remaining ion concentrations (after filtration) subsequently checked by ICP-MS. The exact ion concentrations present in the spiking solutions are outlined in the method section. In particular for Aluminium, concentrations were used in relevant leachable concentration comparable to real-time data obtained from Expansion 51 vials for a relevant placebo formulation as summarized in FIG. 4.

TABLE 2
Visible particles after spiking of (A) myristic acid and (B) lauric acid below
their solubility limit to different anorganic salt solutions (CaCl₂ and NaAlO₃) at
different salt concentrations. Data from. duplicates are presented, which were verified
against the negative controls (without salt) and positive controls (FFA above
solubility limit). Particles were classified as Many particles (>7, xxx), Few particles
(4-7, xx), or Practically free of particles (0-4, /) in E/P box, and Many particles
(>10, xxx), Few particles (6-10, xx), Essentially free of particles (1-5, x), or Free of
particles (0, /) by Seidenader. d = day of inspection. d0 = directly after spiking. *
Nominal salt concentration before pH adjustment and filtration.

| Salt conc. | Ca²⁺ conc. | | E/P | | | | Seidenader | | | |
|---|---|---|---|---|---|---|---|---|---|---|
| (μg/mL) | (μg/mL) | Sample | d0 | d1 | d2 | d7 | d0 | d1 | d2 | d7 |

(A) Myristic acid
CaCl₂ spiking solution

| | | | | | | | | | | |
|---|---|---|---|---|---|---|---|---|---|---|
| 1000 | 360 | 1 | / | xxx | xxx | xxx | xxx | xxx | xxx | xxx |
| | | 2 | / | xxx | xxx | xxx | xxx | xxx | xxx | xxx |
| 500 | 180 | 1 | / | xx | xx | xxx | xxx | xxx | xxx | xxx |
| | | 2 | / | xx | / | xxx | xxx | xxx | xxx | xxx |
| 250 | 90 | 1 | / | / | / | xxx | xxx | xxx | xxx | xxx |
| | | 2 | / | / | / | xxx | xxx | xxx | xxx | xxx |

NaAlO₂ spiking solution

| | | | | | | | | | | |
|---|---|---|---|---|---|---|---|---|---|---|
| 1000 | 0.048 | 1 | / | / | xx | xxx | x | xxx | xxx | xxx |
| | | 2 | / | / | / | xxx | x | xx | x | xxx |
| 500 | 0.024 | 1 | / | / | / | xx | x | xx | x | xxx |
| | | 2 | / | / | / | / | / | x | x | xxx |
| 250 | 0.012 | 1 | / | / | / | / | x | x | x | xxx |
| | | 2 | / | / | / | / | / | x | x | xxx |

(B) Lauric acid
CaCl₂ spiking solution

| | | | | | | | | | | |
|---|---|---|---|---|---|---|---|---|---|---|
| 1000 | 360 | 1 | / | xxx | xx | / | x | xxx | xxx | xxx |
| | | 2 | / | xx | xx | / | / | xx | xxx | xx |
| 500 | 180 | 1 | / | xxx | / | / | / | xxx | xxx | xxx |
| | | 2 | / | / | / | / | / | xx | xxx | xxx |
| 250 | 90 | 1 | / | xxx | / | / | / | xxx | xxx | xxx |
| | | 2 | / | / | / | / | / | xx | xxx | xxx |

NaAlO₂ spiking solution

| | | | | | | | | | | |
|---|---|---|---|---|---|---|---|---|---|---|
| 1000 | 0.048 | 1 | / | / | / | / | x | x | x | xxx |
| | | 2 | / | / | / | / | / | x | / | xxx |
| 500 | 0.024 | 1 | / | / | / | / | x | x | x | x |
| | | 2 | / | / | / | / | x | x | / | / |
| 250 | 0.012 | 1 | / | / | / | / | / | x | x | x |
| | | 2 | / | / | / | / | / | x | x | / |

### Example 2: ‘Real’ Glass Leachables (Mixtures) Lead to FFA Particle Formation

Glass leachables were generated from different types of glass vials, e.g. Exp33 and Exp51 vials, with different matrix solutions including WFI, a glycine solution adjusted to pH 6, and a placebo solution representative for a mAb formulation. Concentrations of glass leachables are provided in Table 1. Defined amounts of myristic and lauric acid below their solubility limit were added to the solutions/mixtures of glass leachables and analyzed. Visible particles in Seidenader are summarized in Table 3 and were detected for all samples in contrast to various controls. Particle formation was dependent on the glass leachable solution and dependent on incubation time. No clear trends were determined for SVP and turbidity at the time points of inspection (d1), however the data suggest an increase in SVP if no visible particles have formed yet. An example for the dependency of particle formation on incubation time is provided in Table 3 for myristic acid in glass leachable solution from Exp51 vials/glycine solution. The example highlights the kinetics of particle formation with no particles directly after spiking and more than 10 particles at incubation day 5 and 7. For selected samples, particles were further characterized and identified by FTIR confirming the presence of free fatty acids. FFA were not confirmed by FTIR for glass leachable solutions generated with WFI, which is linked to the time point of FTIR analysis at d1 and the late onset of particle formation for these samples. Placebo samples were not analyzed further by FTIR as the positive control was found negative attributed to the presence of additional 0.02% PS20, potentially solubilizing the FFA seeds. Characterization of the particles by

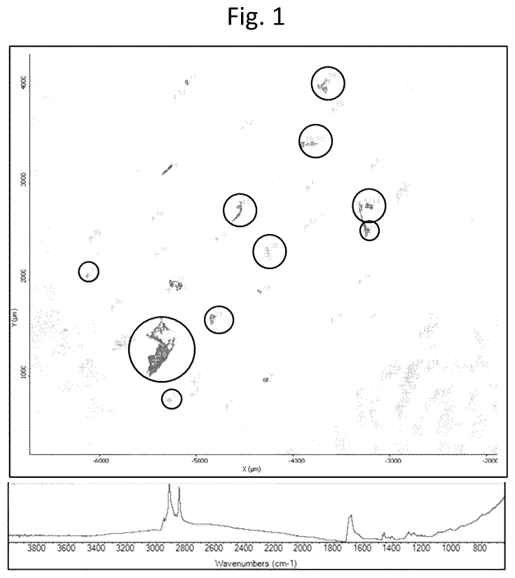

SEM-EDX confirmed the presence of glass leachables, like Aluminium or silicon on the surface of the FFA particles. FIG. 1 shows a typical picture of a gold filter after FTIR analysis highlighting a few FFA particles of different size and a representative FFA spectrum. The spiking study highlights that mixtures of ‘real’ glass leachables lead to precipitation of FFA and particle formation in the visible range dependent on mixture and amount of glass leachables as well as dependent on incubation time.

TABLE 3
Visible particles (Seidenader) and particle identification
of selected samples by FTIR and SEM-EDX (d1).
Visible particles are reported after spiking
of lauric and myristic acid below their
solubility limit to different glass leachable
containing solutions generated by 3
autoclavation cycles in different glass vials.
Results from triplicates are reported in relative
ranking to each other from less to most
particle fromation (+, ++, +++) over
incubation time of up to 7 days. The dependency of
particle formation on incubation time is exemplarily
shown for myristic acid in Exp5 l/glycine matrix,

| | | | | | Glass |
|---|---|---|---|---|---|
| | | | | | leachables |
| Vial | | | Visible | | in SEM- |
| type | Matrix | FFA | particles | FTIR | EDX |
| Exp33 | 3xTS | Myristic | + | n.t. | n.t. |
| vials | WFI | acid | | | |
| | | Lauric acid | + | FFA | + |
| | 3xTS | Myristic | + | n.t. | n.t. |
| | Placebo | acid | | | |
| | | Lauric acid | +++ | n.t. | n.t. |
| | 3xTS | Myristic | + | FFA | + |
| | Glycine | acid | | FFA | + |
| | pH 6 | Lauric acid | | | |
| Exp51 | 3xTS | Myristic | + | n.t. | n.t. |
| vials | WFI | acid | | | |
| | | Lauric acid | ++ | n.t. | n.t. |
| | 3xTS | Myristic | + | n.t. | n.t. |
| | Placebo | acid | | | |
| | | Lauric acid | +++ | n.t. | n.t. |
| | 3xTS | Myristic | +++ | FFA | + |
| | Glycine | acid | | | |
| | pH 6 | Lauric acid | ++ | FFA | + |
| Example for dependency | | | | | |
| on incubation time: | | | Visible particles | | |
| | | Sample no. | d0 | d1 | d5/7 |
| | | 1 | | | >10 |
| | | | | | particles |
| Exp51 | 3xTS | 2 | 1-5 | 1-5 | >10 |
| vials | Glycine | | particles | particles | particles |
| | pH6, | | | | |
| | Myristic | | | | |
| | acid | | | | |
| | | 3 | — | 1-5 | >10 |
| | | | | particles | particles |

n.t. = not tested

### Example 3: Verification of FFA Particle Formation in Presence of Glass Leachables in Aged Matrix (Case Study)

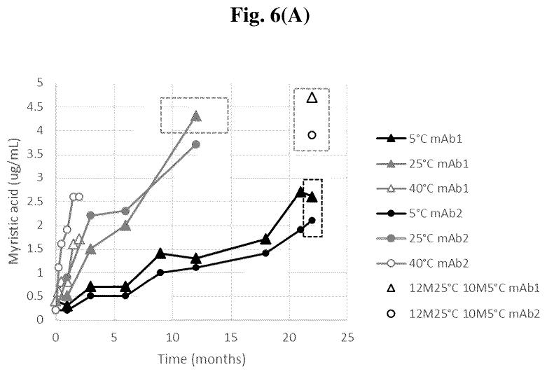

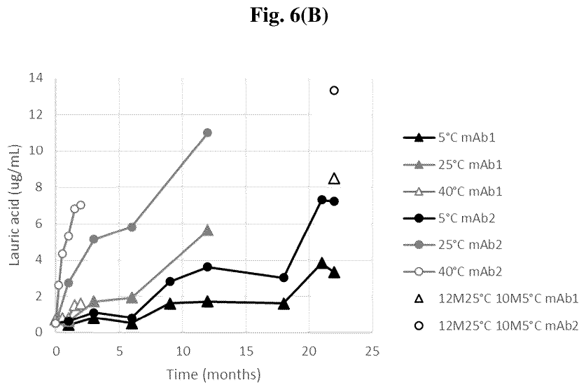

Precipitation of FFA particle in a protein matrix was further studied in aged mAb1 and mAb2 solutions (22M, 5° C.) by addition of different concentrations of ‘real’ glass leachables. In this experiment, the presence of FFA were a result of PS20 degradation forming over shelf-life of the drug product. Mab1 and mAb2 were formulated in the same matrix but differ in the type of mAb (CDRs). Originating from different drug substance processes and purification processes, the PS20 degradation rates were found different (FIG. 5) as well as the type and concentrations of FFAs as a subsequent result (FIG. 6). Interestingly, mAb2 showed visible particles characterized as FFA and Aluminium after 12 M storage at 25° C., whereas mAb1 did not. After 12 M storage at 25° C. plus 10 M storage at 5° C., both formulations showed visible particles identified as a complex of FFA and different glass leachables.

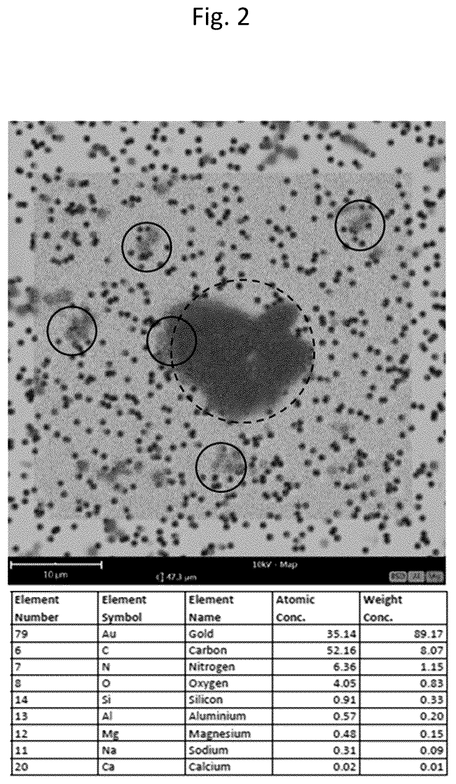

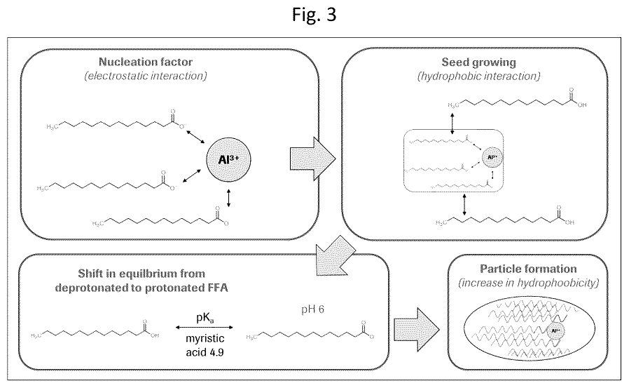

Products were characterized as free of visible particles before the experiment (stored for 22 M at 5° C.). For both formulations, visible particle formation in Seideander inspection machine was observed after incubation with already 50 or 500 μL of different mixtures of glass leachables (Table 4). Results were compared to various controls like the initial time point and in comparison to a spiked placebo solution, which remained free of visible particles. Selected particles were further identified as FFA particles (FTIR) in combination with a mixture of inorganic ions by high resolution SEM-EDX. FIG. 2 shows a representative SEM picture of a FFA particle highlighting the presence of Aluminium and Magnesium. The chemical composition is summarized indicating the presence of a variety of glass leachables. This suggests the precipitation of FFA in presence of the spiked glass leachables acting as nucleation factors. Based on these findings, but without being bound to theory, a potential mechanism of particle formation is illustrated in FIG. 3. FFAs exist in equilibrium of their protonated and deprotonated forms at relevant pH values for biopharmaceuticals. Taking the example of aluminum, triple charged aluminum ions may react with a deprotonated FFA and form highly insoluble aluminum-fatty acid-tri-carboxylates, which would act as nucleating seed. The hydrophobic chain of FFAs may further interact by hydrophobic interaction, fostering seed growing. As displayed in FIG. 3, the proposed mechanism is shown for myristic acid in the presence of aluminum. Finally, particles may precipitate due to increasing hydrophobicity.

TABLE 4
Visible particles (Seidenader). Aged mAb1 and mAb2 formulation
(22M, 5° C.) in comparison to placebo after spiking of different
mixtures and amounts of glass leachables. d = day of inspection

| | Glass leachable's solution | | | Visible particles | | | |
|---|---|---|---|---|---|---|---|
| | | Added volume | | | after spiking/ | | |
| Product | Type | 500 μL | 50 μL | initial | shaking | d1 1 h | d1 4 h |
| Placeob | Glycin 3TS Exp33 | x | — | — | — | — | — |
| | Glycin 3TS Exp33 | — | x | — | — | — | — |
| | WFI 3TS Exp33 | x | — | — | — | — | — |
| | WFI 3TS Exp33 | — | x | — | — | — | — |
| | Glycin 3TS Exp51 | x | — | — | — | — | — |
| | Glycin 3TS Exp51 | — | x | — | — | — | — |
| | CaCl₂ 1 mg/mL | x | — | — | — | — | — |
| | NaAlO₂ pH 6 | x | — | — | — | — | — |
| mAb1 | Glycin 3TS Exp33 | x | — | — | + | + | + |
| | Glycin 3TS Exp33 | — | x | — | + | — | + |
| | WFI 3TS Exp33 | x | — | — | + | + | + |
| | WFI 3TS Exp33 | — | x | — | — | + | — |
| | Glycin 3TS Exp51 | x | — | — | + | + | + |
| | Glycin 3TS Exp51 | — | x | — | + | + | + |
| | CaCl₂ 1 mg/mL | x | — | — | + | + | + |
| | NaAlO₂ pH 6 | x | — | — | + | + | + |
| mAb2 | Glycin 3TS Exp33 | x | — | — | — | + | — |
| | Glycin 3TS Exp33 | — | x | — | + | + | + |
| | WFI 3TS Exp33 | x | — | — | + | + | + |
| | WFI 3TS Exp33 | — | x | — | + | + | + |
| | Glycin 3TS Exp51 | x | — | — | + | + | + |
| | Glycin 3TS Exp51 | — | x | — | + | + | + |
| | CaCl₂1 mg/mL | x | — | — | + | + | + |
| | NaAlO₂ pH 6 | x | — | — | + | + | + |

### Example 4: Influence of Washing and Sterilization Procedures on Nucleation of Particles as a Result of Polysorbate Degradation

Preparation of Glass Vials Expansion 51 glass vials (Fiolax®) in the 20cc configuration were purchased from Schott North America Inc. (NY, USA). The vials comply with type I glass as per European Pharmacopoeia (Ph. Eur.). After washing and depyrogenation as described below, vials were filled with 12.2 mL of a placebo buffer (20 mM His/His-HCl, 240 mM sucrose, 10 mM methionine, pH 5.5) containing (1.) no further excipients (negative control, NC), (2.) 0.4 mg/mL polysorbate 20 (PS20) or (3.) 0.4 mg/mL PS20 with additionally spiked AlCl₃ to a final Al³⁺ concentration of ˜250 ppb (positive control, PC). Beforehand, the 0.4 mg/mL PS20 was degraded by ˜10% with C. Antarctica-coupled beads as described previously¹³. Vials were stored upright at 5° C., 25° C./60% RH, and 40° C./70% RH.

### Washing and Depyrogenation

Vials were washed with water for injection (WFI) at 70° C. (water and air pressure 1 bar, final air pressure 2.5 bar) using a FAW1020 vial washing machine (Bausch & Stroebel, Germany). Subsequently, vials were placed in stainless steel boxes and dried for 96 h under laminar air flow.

Vials were further processed according to either best or worst case sterilization conditions as specified in Table 5. Depyrogenation of all vials was conducted in a DHT2550 sterilization tunnel (Bausch & Stroebel, Germany). The process conditions differ in presence of residual moisture, heating zone temperature, sterilization temperature, residence time in the tunnel and conveyer belt speed. Vials processed according to the worst case conditions were filled with 281 μL of WFI prior to depyrogenation to simulate the presence of residual moisture. In addition, the conveyor belt was stopped after the vials had entered the sterilization zone for prolonged residence time in the tunnel. The time in the heating, sterilization, and cooling zone was calculated to be 41 min, 43.5 min, and 50 min, respectively, for the best case conditions (134.5 min total), compared to 3 min, 16 h, and 3.5 min, respectively, for the worst case conditions (966.5 min total).

TABLE 5
Depyrogenation process parameters

| | | Temperature | Conveyor | | | |
|---|---|---|---|---|---|---|
| Conditions | Residual | of heating | speed | Sterilization | Residence | |
| reffered | moisture | zone | (entry) | Temperature | time in | Conveyor speed |
| to as: | (μL) | (° C.) | (mm/min) | (° C.) | tunnel (h) | (out) (mm/min) |
| Best case | — | 60 | 20 | 180 | — | 20 |
| | | | (min. 5) | | | (min. 5) |
| Worst case | 281* | 45 | 280 | 350 | 16 ± 1 | 280 |
| | | | (max. 300) | | | (max. 300) |

*volume normalized to surface area of 2 cc vials with 80 μL residual moisture as default. Normalized volume = surface area * F; with F = 80 μL / surface area of 2 cc vial

### Visual Inspection

Particles were identified by visual inspection using both a black/white panel according to Ph. Eur. 2.9.20 and a Seidenader V 90-T instrument (Seidenader Maschinenbau GmbH, Markt Schwaben, Germany). The latter is referred to as enhanced visual inspection in this study. For enhanced visual inspection, samples were illuminated from behind as well as from the bottom and the top. Containers were rotated and inspected through 2-fold magnifying glass. For both instruments, samples were inspected after allowing the containers to equilibrate to room temperature for 3 h. The number of particles as mean of 5 vials is reported.

### Results

Particle formation was observed for worst case sterilized samples after storage at 25° C. and 40° C. starting from 2 months on when analyzed by enhanced visual inspection (Table 6A). For best case samples, particle formation starts at 40° C. after 3months, but to a smaller extent. Given that particle formation is a stochastic event, trends from analysis by E/P box (Table 6B) follow the results from enhanced visual inspection, which is more sensitive. Particle formation started for the 40° C. worst case sterilized vials after 3 months. In general, no particle formation was observed at 5° C. storage up to 3 months for none of the visual inspection methods and samples tested. Negative controls have been confirmed for absence of particles and positive controls have been confirmed for presence of particles using both visual inspection methods, for all temperatures, and time points of analysis.

It can thus be concluded that the washing and sterilization procedures has a major influence on nucleation of particles as a result of polysorbate degradation.

TABLE 6
Summary of visual inspection results.

| | | | | | | |
|---|---|---|---|---|---|---|
| Sterilization | Storage | | | | | |
| process | temperature | Sample | Initial | 1M | 2M | 3M |

(A) Enhanced visual inspection (Seidenader)

| | | | | | | |
|---|---|---|---|---|---|---|
| Best case | 5° C. | NC | 0 | 0 | 0 | 0 |
| | | PC | >10 | >10 | >10 | >10 |
| | | sample | 0 | 0 | 0 | 0 |
| | 25° C. | NC | 0 | 0 | 0 | 0 |
| | | PC | >10 | >10 | >10 | >10 |
| | | sample | 0 | 2 | 0 | 0 |
| | 40° C. | NC | 0 | 0 | 0 | 0 |
| | | PC | >10 | >10 | >10 | >10 |
| | | sample | 0 | 1 | 0 | 6 |
| Worst Case | 5° C. | NC | 0 | 0 | 0 | 0 |
| | | PC | >10 | >10 | >10 | >10 |
| | | sample | 0 | 0 | 0 | 2 |
| | 25° C. | NC | 0 | 0 | 0 | 0 |
| | | PC | >10 | >10 | >10 | >10 |
| | | sample | 0 | 2 | 8 | 6 |
| | 40° C. | NC | 0 | 0 | 0 | 0 |
| | | PC | >10 | >10 | >10 | >10 |
| | | sample | 0 | 4 | >10 | >10 |

(B) Visual inspection (Ph. Eur.)

| | | | | | | |
|---|---|---|---|---|---|---|
| Best case | 5° C. | NC | 0 | 0 | 0 | 0 |
| | | PC | 2 | >7 | >7 | >7 |
| | | sample | 0 | 0 | 0 | 0 |
| | 25° C. | NC | 0 | 0 | 0 | 0 |
| | | PC | 3 | >7 | >7 | >7 |
| | | sample | 0 | 0 | 0 | 1 |
| | 40° C. | NC | 0 | 0 | 0 | 0 |
| | | PC | >7 | >7 | >7 | >7 |
| | | sample | 0 | 0 | 0 | >7 |
| Worst Case | 5° C. | NC | 0 | 0 | 0 | 0 |
| | | PC | 0 | >7 | >7 | >7 |
| | | sample | 0 | 0 | 0 | 1 |
| | 25° C. | NC | 0 | 0 | 0 | 0 |
| | | PC | 2 | >7 | >7 | >7 |
| | | sample | 0 | 0 | 1 | >7 |
| | 40° C. | NC | 0 | 0 | 0 | 0 |
| | | PC | >7 | >7 | >7 | >7 |
| | | sample | 0 | 0 | >7 | >7 |

### REFERENCES

(1) Kishore RS. Part II: Challenges with Excipients—Polysorbate Degradation and Quality, in Challenges in Protein Product Development. AAPS Advances in the Pharmaceutical Sciences Series 38. Warne N and Mahler HC eds. Springer 2018, Switzerland. pp. 25-62.
(2) Khan TA, Mahler HC, Kishore RS. Key interactions of surfactants in therapeutic protein formulations: A review. Eur J Pharm Biopharm, 2015. 97: 60-7.
(3) US Food and Drug Administration: Highlights of prescribing information for ( . . . ) VARUBI® (rolapitant) injectable emulsion, for intravenous use. https://www.accessdata.fda.gov/drugsatfda_docs/label/2017/208399s000lbl.pdf (29 Apr. 2019)
(4) Kerwin BA. Polysorbates 20 and 80 Used in the Formulation of Protein Biotherapeutics: Structure and Degradation Pathways. J Pharm Sci, 2008. 97(8), 2924-2935.
(5) Ditter D, Mahler HC, Roehl H, Wahl M, Huwyler J, Nieto A, Allmendinger A. Charaterization of surface properties of glass vials used as primary packaging material for parenterals. Eu J Pharm Biopharm, 2018. 125:58-67.
(6) Doshi N, Demeule B, Yadav S. Understanding Particle Formation: Solubility of Free Fatty Acid as Polysorbate 20 Degradation Byproducts in Therapeutic Monoclonal Antibody Formulations. Mol Pharm, 2015. 12(11): 3792-804.
(7) Honemann M, Wendler J, Graf T, Bathke A, Bell CH. Monitoring polysorbate hydrolysis in biopharmaceuticals using a QC-ready free fatty acid quantification method. J Chromatogr B Analyt Technol Biomed Life Sci. 2019 Mar. 26;1116:1-8.
(8) Ditter D, Nieto A, Mahler HC, Roehl H, Wahl M, Huwyler J, Allmendinger A. Evaluation of Glass Delamination Risk in Pharmaceutical 10 mL/10 R Vials. J Pharm Sci, 2018. 107(2):624-637.
(9) Ditter D, Mahler HC, Gohlke L, Nieto A, Rochl A, Huwyler J, Wahl M, Allmendinger A. Impact of Vial Washing and Depyrogenation on Surface Properties and Delamination Risk of Glass Vials. Pharm Res, 2018. 35(7):146.
(10) European Pharmacopeia 9.4, 2019. Particulate contamination: Visible particles [2.9.20]
(11) European Pharmacopeia 9.4, 2019. Particulate contamination: Subvisible particles [2.9.19]
(12)European Pharmacopeia 9.4, 2019. Clarity and degree of opalescence of liquids [2.2.1]
(13) Graf et al. Controlled polysorbate 20 hydrolysis—A new approach to assess the impact of polysorbate 20 degradation on biopharmaceutical product quality in shortened time. Eu J Pharm Biopharm. 2020 (152) pp. 318-326

1. A stable aqueous composition comprising a protein together with pharmaceutically acceptable excipients such as, for example, buffers, stabilizers including antioxidants, and surfactants, wherein said composition further contains a mixture of one or several types of inorganic ions diffused out of the packaging material, such as a glass vial, and substances resulting from the degradation of said surfactants without forming visible particles.

2. A composition according to claim 2, wherein said inorganic ions are selected from Aluminium, Boron, Silicon, Calcium, Magnesium, Potassium, and Sodium.

3. A composition according to any one of claim 1 or 2, wherein the pH of said composition is in the range of 5 to 7, preferably around 6.

4. A composition according to any one of claims 1 to 3, wherein the protein is an antibody, preferably a monoclonal antibody.

5. A composition according to any one of claims 1 to 4, comprising a concentration of up to 0.03 μg/ml aluminium, and/or up to 0.05 μg/ml boron, and/or up to 0.5 μg/ml silicon.

6. A composition according to any one of claims 1 to 5, wherein the stabilizer is selected from the group consisting of sugars, sugar alcohols, sugar derivatives, or amino acids.

7. A composition according to any one of claims 1 to 6, wherein the buffer is selected from the group consisting of acetate, succinate, citrate, arginine, histidine, phosphate, Tris, glycine, aspartate, and glutamate buffer systems.

8. A composition according to any one of claims 1 to 7, wherein the surfactant is selected from the group consisting of non-ionic surfactants, preferably polysorbates.

9. A composition according to any one of claims 1 to 8, wherein the substances resulting from the degradation of said surfactants are free fatty acids, below their solubility level in water at room temperature.

10. A composition according to any one of claims 1 to 9, wherein the pharmaceutically acceptable excipients are: 1000 U/mL hyaluronidase in 20 mM HisHCl buffer pH 5.5, 105 mM Trehalose, 100 mM Sucrose, 10 mM Methionine, and 0.04% Polysorbate 20.

11. A method for obtaining a composition according to any one of claims 1 to 10, wherein said method comprises selecting a primary packaging material which prevents leaching of one or several of the inorganic ions as defined in claim 1 into said composition.

12. The method according to claim 11, wherein said primary packaging material is a glass or a polymer vial.

13. The method according to claim 11 or 12, further comprising the steps of a) washing and/or b) depyrogenation of the primary packaging material prior to its use.

14. The method according to any one of claims 11 to 13, wherein said method provides stability of said composition against the formation of visible particles.

15. A pharmaceutical dosage form comprising a composition according to any one of claims 1 to 10 in a container, wherein the concentration of one or several inorganic ions selected from Aluminium, Boron, Silicon, Calcium, Magnesium, Potassium, and Sodium in that composition remains substantially constant during the time of its authorized shelf life.
